SEQUENCE LISTING (3 sequences)

SEQ ID NO: 1 - DNA, 1335 bp, Chilo iridescent virus
gaagtttaaatttttaactataaacatttttaatgacacaaaatggatcttaaagacgaatttattcaaataattaaaaagtattctgaactttctcttaaggaatctgaaaacgaaacaaaaaaatatatacaagaagtatttaacgtatcgttgtctgaagtatttataaaagaaccatcaaaacaggaaacattaaaacaggaaccaactcaagaaaccaatggatgtatttatatttttaaaaaaggaaaaaatataggacaaaaatgtggttctggaaaatctaaattttgttataaacataaaaagaatgatttaaatataaaagtgaatcaagaaagaaatattaaaaaacctataccgaaacctataccaacaaacaaaattgaaaccggatcagttatcaatcagaattggtttataggaacttcaataggtaagggaggttttggagaaatatattcggctgctaaatttaatgatcatggtaactacaaagacgacgattttagttttgcaattaaaatcgaacccaaaagtaatggacccttatttgttgaaatgcatttttacaaacgcgttattgtagaaaaagaaattgaaaaatttaaacttcaaaaaaatattcaatatttaggattacctaaatattatggatctggattgtacaatgattatagatatattgttatggaaaagtatgattcaaacattgataagttgtttagaaatggtgacttaaattcttctacaattttacaaataggaattcaaatattaaacattgtggagtacatccattctaaaggatatattcatgctgacattaagggcgaaaatatactattgaaagaaaatgatacttctcatatttatttagttgattttggattatgttctcgttatagtaacatttatcaaccagatcctaaaaaagcacataatggtacattaaaatatacaagcagagatagccatcaaggtgttgaatcaagaagaggagatttagaaattctaggctataatatgatagagtggtctggtggtattttaccatggcaacatttatgtaaaaagagtgctggaaaaaagattttaaacgaagtttcagaaagtaaaataaaatatatgaatgatttatctttgttttttaacaaagttcaatttaacgacaccaatttaaaaaataaattacaaacgtattttgaaaccatcatcaaaataacatttgaagaattaccaccttatcaactattacataacatttttaactaacatatttaactaatttttattttaatggacaatgtccattaaaataaataagaggag

SEQ ID NO: 2 - protein, 379 aa, Chilo iridescent virus
MDLKDEFIQIIKKYSELSLKESENETKKYIQEVFNVSLSEVFIKEPSKQETLKQEPTQETGCIYIFKKGKNIGQKCGSGKSKFCYKHKKNDLNIKVNQERNIKKPIPKPIPTNKIETGSVINQNWFIGTSIGKGGFGEIYSAAKFNDHGNYKDDDFSFAIKIEPKSGPLFVEMHFYKRVIVEKEIEKFKLQKNIQYLGLPKYYGSGLYNDYRYIVMEKYDSNIDKLFRNGDLNSSTILQIGIQILNIVEYIHSKGYIHADIKGENILLKENDTSHIYLVDFGLCSRYSNGDLEILGYNMIEWSGGILPWQHLCKKSAGKKILNEVSESKIKYMNLSLFFNKVQFNDTNLKNKLQTYFETIIKITFEELPPYQLLHNIFN

SEQ ID NO: 3 - DNA, 876 bp, Chilo iridescent virus
atggatcttaaagacgaatttattcaaataattaaaaagtattctgaactttctcttaaggaatctgaaaacgaaacaaaaaaatatatacaagaagtatttaacgtatcgttgtctgaagtatttataaaagaaccatcaaaacaggaaacattaaaacaggaaccaactcaagaaaccaatggatgtatttatatttttaaaaaaggaaaaaatataggacaaaaatgtggttctggaaaatctaaattttgttataaacataaaaagaatgatttaaatataaaagtgaatcaagaaagaaatattaaaaaacctataccgaaacctataccaacaaacaaaattgaaaccggatcagttatcaatcagaattggtttataggaacttcaataggtaagggaggttttggagaaatatattcggctgctaaatttaatgatcatggtaactacaaagacgacgattttagttttgcaattaaaatcgaacccaaaagtaatggacccttatttgttgaaatgcatttttacaaacgcgttattgtagaaaaagaaattgaaaaatttaaacttcaaaaaaatattcaatatttaggattacctaaatattatggatctggattgtacaatgattatagatatattgttatggaaaagtatgattcaaacattgataagttgtttagaaatggtgacttaaattcttctacaattttacaaataggaattcaaatattaaacattgtggagtacatccattctaaaggatatattcatgctgacattaagggcgaaaatatactattgaaagaaaatgatacttctcatatttatttagttgattttggattatgttctcgttatagtaacatt

(19) United States
(12) Patent Application Publication
(10) Pub. No.: US 2009/0069239 A1
(43) Pub. Date: Mar. 12, 2009

(54) Use of Iridoptin to Induce Toxicity in Insects

(75) Inventor: Shan L. BILIMORIA, Lubbock, TX (US)

Correspondence Address: KAMMER BROWNING PLLC, 7700 BROADWAY, SUITE 202, SAN ANTONIO, TX, 78209, US

(73) Assignee: TEXAS TECH UNIVERSITY, Lubbock, TX (US)

(21) Appl. No.: 12/205,857

(22) Filed: Sep. 6, 2008

Related U.S. Application Data
(60) Provisional application No. 60/970,489, filed on Sep. 6, 2007.

(51) Int. Cl. A01N 37/18; C07H 21/00; C07K 2/00; C07K 1/107; C12P 19/30; C12N 15/63; C12N 5/10; A01P 7/04; A01N 43/90; C12N 1/21; C12N 15/82; C12N 15/74; A01H 1/00; A01H 5/00

(52) U.S. Cl. 514/12; 536/23.2; 530/300; 530/402; 435/89; 435/320.1; 435/419; 435/243; 435/468; 435/471; 800/279; 800/302; 514/44

(57) ABSTRACT

Improved methods and compounds to control insects, involving a biological control method to induce toxicity in targeted insects using iridoptin. The present invention induces high levels of apoptosis and inhibition of host protein synthesis in insect cells. It is the first viral toxin against non-lepidopteran insects and is distinct from existing bacterial toxins, such as Bacillus thuringiensis toxins, which are not effective against most beetles, including the boll weevil, and the Baculoviridae, which is the main group of viruses currently used as biological control agents. Iridoptin will have use in the control of agricultural pests. It will increase productivity and reduce disease transfer by vectors and household pests. By extension it has application in cancer therapy and other medical treatments where apoptosis is critical to removal of certain cells.

### CROSS REFERENCE TO RELATED APPLICATION

This application claims the benefit under Title 35 United States Code § 119(e) of U.S. Provisional Application No. 60/970,489; Filed: Sep. 6, 2007, the full disclosure of which is incorporated herein by reference.

### FIELD OF THE INVENTION

The present invention relates to an improved method of inducing toxicity in insect cells by protein engineering. More specifically, the present invention relates to use of iridoptin, a high activity cleaved polypeptide derived from the istk gene product, used to induce high levels of apoptosis and inhibition of host protein synthesis in insect cells and mortality in aphids.

### BACKGROUND OF THE INVENTION

Without limiting the scope of the disclosed method, the background is described in connection with an improved method of inducing toxicity in insect cells by protein engineering.

The economic impact of aphids, lygus bug, silver leaf whiteflies, boll weevils, and noctuids is estimated at $3.7 billion annually for the U.S. and $400 million for Texas. These insects contribute to increased water demand and cause billions of dollars of agricultural damage.

Eradication programs and chemical control have limitations; for instance, chemical control requires increasingly higher doses to be effective. These higher doses have adverse side effects on beneficial insects and groundwater. An improved biological control method is needed. Therefore, transgenic pest-resistant crops and insecticidal microbes are critically needed. The identification and development of toxin genes are essential for implementing such approaches.

U.S. Pat. No. 6,200,561 (the '561 patent) issued to the present inventor in 2001, discloses the use of viral proteins for controlling the cotton boll weevil and other insect pests. The full disclosure of U.S. Pat. No. 6,200,561, entitled Use of Viral Proteins for Controlling the Cotton Boll Weevil and Other Insect Pests is incorporated herein in its entirety by reference. The '561 patent involves a Chilo iridescent virus (CIV; New Zealand strain) capsid protein extract that kills neonate larvae, inhibits host expression and induces apoptosis. CIV causes infection in several orders of insects. The present invention discloses the use of a composition identified as iridoptin, which is even more efficient in inducing apoptosis and inhibition of host protein synthesis in insects and which also induces mortality in aphids.

### SUMMARY OF THE INVENTION

The present invention, therefore, provides an improved means to control insects, involving a biological control method to induce toxicity in targeted insects using iridoptin. It is the first viral toxin against non-lepidopteran insects and is distinct from existing bacterial toxins, such as Bacillus thuringiensis toxins, which are not effective against aphids or most beetles, and the Baculoviridae, which is the main group of viruses currently used as biological control agents. Iridoptin finds specific use in the control of agricultural pests. Iridoptin can serve to increase agricultural productivity and reduce disease transfer by vectors and household pests. By extension, iridoptin finds application in cancer therapy and other medical treatments where apoptosis is critical to removal of certain cells.

In summary, the present invention discloses an improved method for inducing toxicity in insect cells and in aphids by protein engineering. More specifically, the disclosed method can be used to induce high levels of apoptosis and inhibition of host protein synthesis in insect cells, as well as mortality in aphid populations.

### BRIEF DESCRIPTION OF THE DRAWINGS

For a more complete understanding of the features and advantages of the present invention, reference is now made to the detailed description of the invention along with the accompanying figures in which:

FIG. 1A is a depiction of the location of the istk gene sequence in the CIV genome in accordance with embodiments of the disclosure;

FIG. 1B is a depiction of the nucleotide sequence of the 2-kbp region encoding the complete istk gene (SEQ ID NO: 1);

FIG. 1C is a depiction of the nucleotide sequence of the istk gene in the CIV genome and the subgenic fragment (underlined) of the istk gene which codes for iridoptin;

FIG. 2 is a depiction of the cleaved polypeptide from the ISTK product in accordance with embodiments of the disclosure;

FIG. 3 is an electrophoretic analysis of the iridoptin product in accordance with embodiments of the disclosure;

FIG. 4A is a depiction of apoptosis in iridoptin-exposed budworm cells (CF) in accordance with embodiments of the disclosure;

FIG. 4B is a depiction of apoptosis in actinomycin D-exposed budworm cells (CF) in accordance with embodiments of the disclosure;

FIG. 4C is a depiction of apoptosis in heat-inactivated iridoptin-exposed budworm cells (CF) in accordance with embodiments of the disclosure;

FIG. 4D is a depiction of apoptosis in control buffer-exposed budworm cells (CF) in accordance with embodiments of the disclosure;

FIG. 5A is a depiction of apoptosis in iridoptin-exposed boll weevil cells (AG) in accordance with embodiments of the disclosure;

FIG. 5B is a depiction of apoptosis in heat-inactivated iridoptin-exposed boll weevil cells (AG) in accordance with embodiments of the disclosure;

FIG. 5C is a depiction of apoptosis in actinomycin D-exposed boll weevil cells (AG) in accordance with embodiments of the disclosure;

FIG. 5D is a depiction of apoptosis in control buffer-exposed boll weevil cells (AG) in accordance with embodiments of the disclosure;

FIG. 5E is a TUNEL assay on iridoptin-induced (10 μg/ml) apoptosis in AG cells in accordance with embodiments of the disclosure;

FIG. 5F is a TUNEL assay on iridoptin-induced (20 μg/ml) apoptosis in AG cells in accordance with embodiments of the disclosure;

FIG. 5G is a TUNEL assay on Actinomycin D-induced apoptosis in AG cells in accordance with embodiments of the disclosure;

FIG. 5H is a TUNEL assay on control buffer-induced apoptosis in AG cells in accordance with embodiments of the disclosure;

FIG. 5J is a dose-response analysis of iridoptin-induced apoptosis in AG cells in accordance with embodiments of the disclosure;

FIG. 6A is a depiction of iridoptin-induced inhibition of protein synthesis in boll weevil cells in accordance with embodiments of the disclosure;

FIG. 6B is a depiction of quantitative iridoptin-induced inhibition of protein synthesis in boll weevil (AG) in accordance with embodiments of the disclosure;

FIG. 6C is a depiction of dose-response analysis on iridoptin-induced inhibition of protein synthesis in boll weevil (AG) cells in accordance with embodiments of the disclosure;

FIG. 7 is a depiction of iridoptin-induced inhibition of protein synthesis in budworm (CF) cells in accordance with embodiments of the disclosure;

FIG. 8 is a depiction of iridoptin-induced mortality of aphids in accordance with embodiments of the invention.

### DETAILED DESCRIPTION OF THE PREFERRED EMBODIMENTS

Disclosed herein is an improved method of inducing toxicity in insect cells by protein engineering, wherein insect cells are exposed to iridoptin which then induces apoptosis, inhibits host protein synthesis in insect cells as well as mortality in aphid populations. The numerous innovative teachings of the present invention will be described with particular reference to several embodiments (by way of example, and not of limitation).

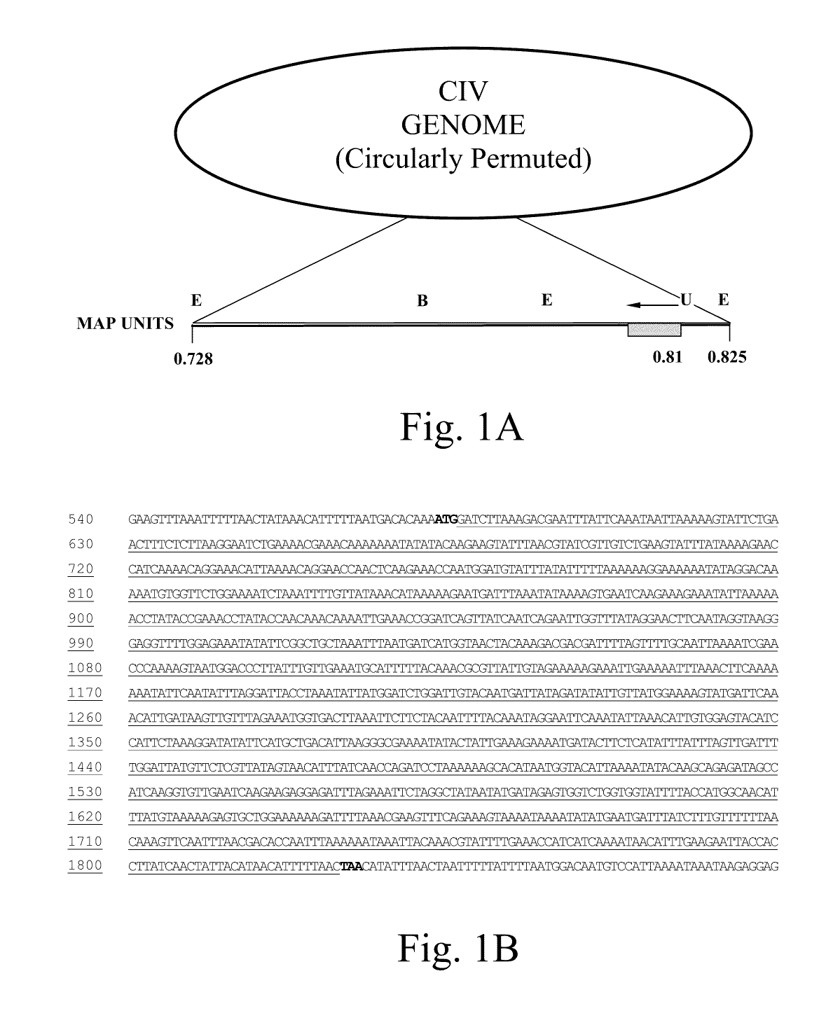

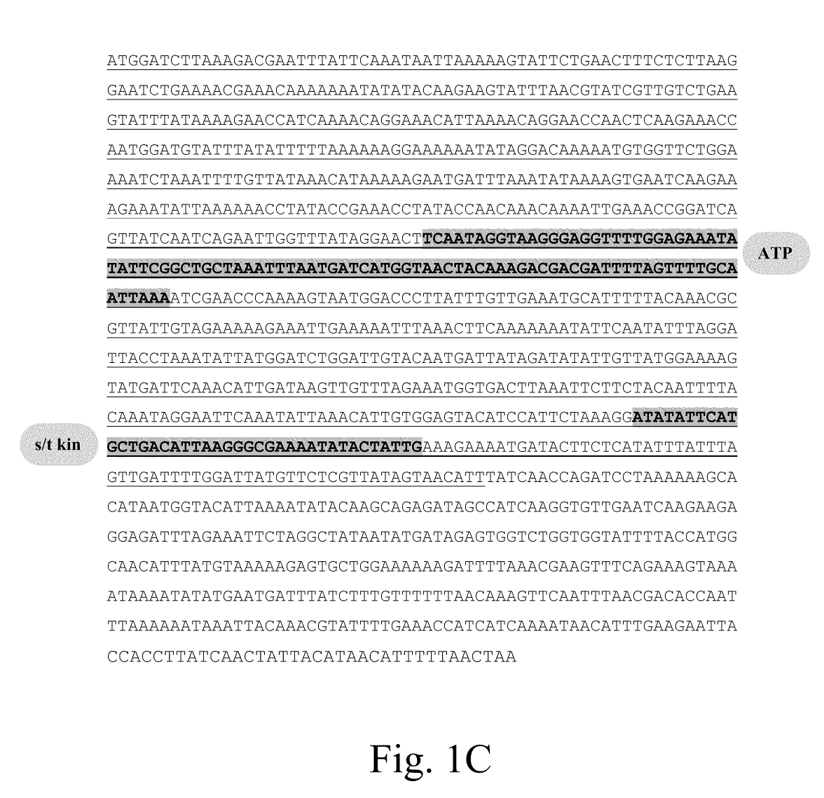

Reference is first made to FIGS. 1A, 1B, and 1C wherein a region of the istk gene in Chilo iridescent virus (CIV; New Zealand strain) DNA was mapped and sequenced and an open reading frame for a serine/threonine kinase was identified. The open reading frame encoding the putative istk gene was identified by primer walking and mapped to a 2-kbp region (shown in FIG. 1A by a filled-in grey block) spanning the Eco RI sites (“E”) separating fragments B and U in the CIV genome. This gene was shown to be active and was designated istk (iridovirus serine threonine kinase). FIG. 1B depicts the nucleotide sequence of the 2-kbp region encoding the complete 1236-bp istk gene (shown underlined in FIG. 1B) which was determined. The gene product (ISTK) is a 49-kDa polypeptide. The start and stop sites for this gene are indicated in bold in FIG. 1B.

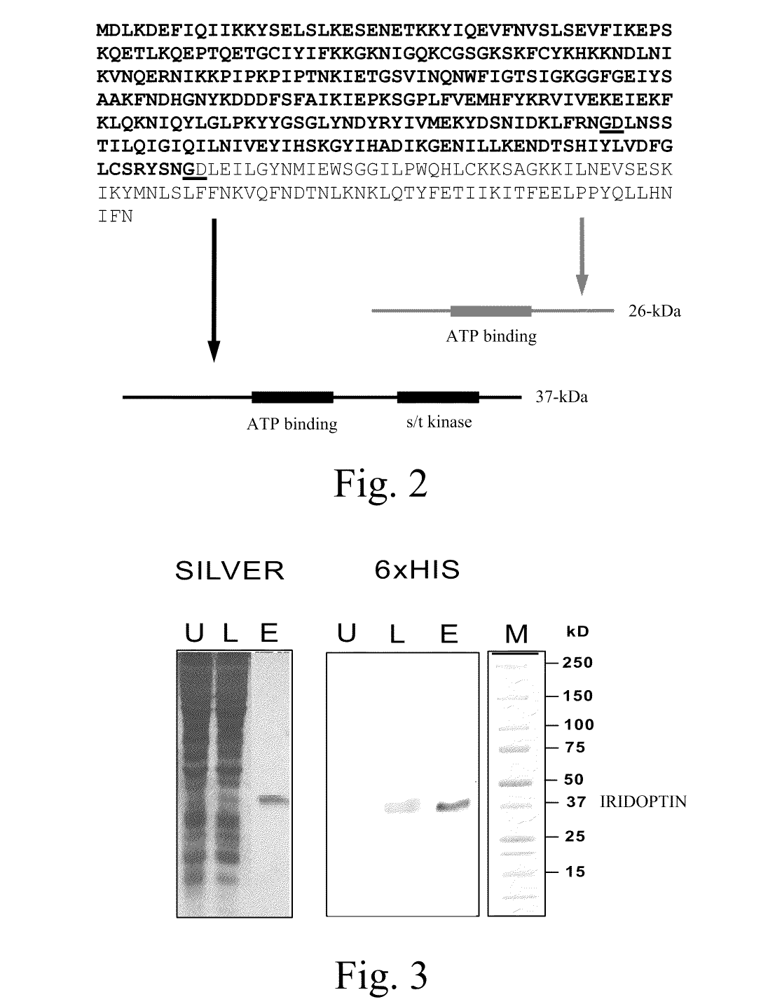

Reference is now made to FIG. 2, wherein a cleaved 37-kDa polypeptide resulting from ISTK was analyzed for C-terminal sequence. As indicated above, prior U.S. Pat. No. 6,200,561 identified a Chilo iridescent virus capsid protein extract that killed neonate boll weevil larvae, inhibited host expression and induced apoptosis. Further research has shown that CIV contains a serine-threonine kinase enzyme. The gene has now been cloned and expressed for this enzyme and it has been demonstrated that the 49-kDa product (ISTK) induces apoptosis in 63% of treated insect cells and necrosis in the remaining cells. Apoptosis was detected by DNA fragmentation, blebbing, and with the TUNEL assay. The inventor has now made the significant finding that a 37-kDa cleavage product of the ISTK 49-kDa protein induces an apoptotic effect in nearly all cells in the population. Thus, no additional factors or gene products are required for a complete effect, and post-translational processing of the 49-kDa gene product is important for activity. The new polypeptide was necessary and sufficient for full toxic effect. Dose response studies using blebbing assays in CF and AG cells indicated that 100 ng/ml and 900 ng/ml, respectively, of the 37-kDa polypeptide were required to induce apoptosis in 50% of the cell population.

In the present invention, the cleavage site on ISTK has been identified and this information has been used to tailor a subgenic fragment of the istk gene (shown underlined in FIG. 1C). The positions of the s/t kinase domain and the ATP binding site are shown highlighted in FIG. 1C. The subgenomic fragment (underlined) from the istk gene sequence was cloned in-frame with the C-terminal polyhistidine tag of Pichia expression vector (pPICZ C). The stop codon after polyhistidine tag in the expression vector was utilized for termination. The present invention further discloses that the modified gene codes for a 37-kDa polypeptide now designated iridoptin, which is more efficient in inducing apoptosis and inhibition of host protein synthesis than the product from the original gene.

FIG. 2 depicts the C-terminal sequencing of 37-kDa polypeptide following carboxypeptidase Y digest which was carried out by the Macromolecular Structure, Sequencing and Synthesis Facility, Department of Biochemistry at Michigan State University. The C-terminal amino acids were asparagine-glycine (Asn-Gly, or N-G). FIG. 2 shows that N-G was detected at two sites (underlined in FIG. 2) in the amino acid sequence derived from corresponding regions of the CIV istk gene. Based on this sequence, cleavage should occur at a glycine-aspartate (Gly-Asp, G-D) site. Cleavage at the amino-terminal G-D residues should result in the formation of a 26-kDa polypeptide containing only the ATP binding site. Cleavage at the carboxy-terminal G-D site should yield the predicted 37-kDa polypeptide with both ATP binding site and s/t kinase motif.

MALDI-TOF analysis (matrix-assisted laser desorption/ionization-time of flight mass spectrometry) conducted at the Institute for Cellular and Molecular Biology Core Facility, University of Texas, Austin) confirmed the identity of the 37-kDa polypeptide and the presence of the ATP-binding and s/t kinase motifs. The subgenic DNA sequence coding for the 37-kDa polypeptide was amplified by PCR using specifically designed primers and CIV genomic DNA and expressed in the Pichia system (Invitrogen) to yield 6×His-tagged product.

Reference is now made to FIG. 3, wherein it is shown that the yeast lysates from Pichia expression contained His-tagged 37-kDa product as detected by silver staining of polyacrylamide gels and western blotting. Purification of this material through nickel columns (ProBond, Invitrogen, CA) that bind 6×His-tagged polypeptides yielded pure 37-kDa polypeptide; i.e., iridoptin as detected with 6×His antibody probe and silver staining for total protein. FIG. 3 shows analysis of iridoptin product expressed in the Pichia system and purified by affinity chromatography with the silver-stained gel and western blot (using 6×His antibody probe). As shown in FIG. 3, the columns under “U” show that uninduced yeast lysate did not express iridoptin. The columns under “L” show that the induced yeast lysate reveals the predicted iridoptin band. “E” shows iridoptin purified on His-binding nickel affinity column (ProBond) retained the 6×His tag and was detectable as a pure polypeptide by silver staining and western blotting. Column “M” shows the molecular weight markers, wherein the numbers represent molecular mass in kilo-Daltons.

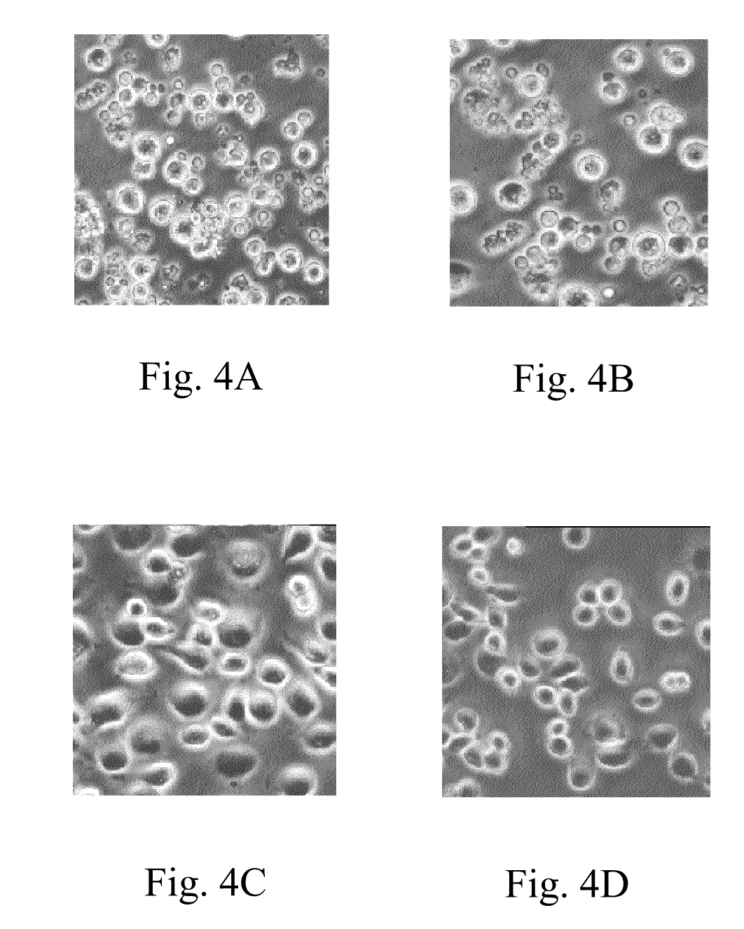

Reference is now made to FIGS. 4A, 4B, 4C, and 4D wherein it is shown that iridoptin induces apoptosis in spruce budworm (CF124T) cells. Cells in Nunc 60-well trays (5.6×10³ cells per well in 7 μl medium) were treated and incubated at 28° C. and examined for apoptotic blebbing by phase-contrast microscopy at 24 hr post treatment. The iridoptin product induced apoptotic blebbing in more than 90% of CF cells compared to 98% in positive controls treated with actinomycin D. Results are demonstrated in FIG. 4A showing Iridoptin: 37-kDa polypeptide expressed from subgenic istk (Pichia; 10 μg/ml); 92% blebbing, SD 4.7. FIG. 4B showing actinomycin D (4 μg/ml; positive control); 98% blebbing, SD 1.1. FIG. 4C showing Δ Iridoptin (heat-inactivated iridoptin 10 μg/ml, 65° C., 30 min) 5% blebbing. FIG. 4D showing mock treatment with buffer (1× Rinaldini's Balanced Salt Solution; RBSS), 7% blebbing. Magnification: 800× in FIGS. 4A-4D.

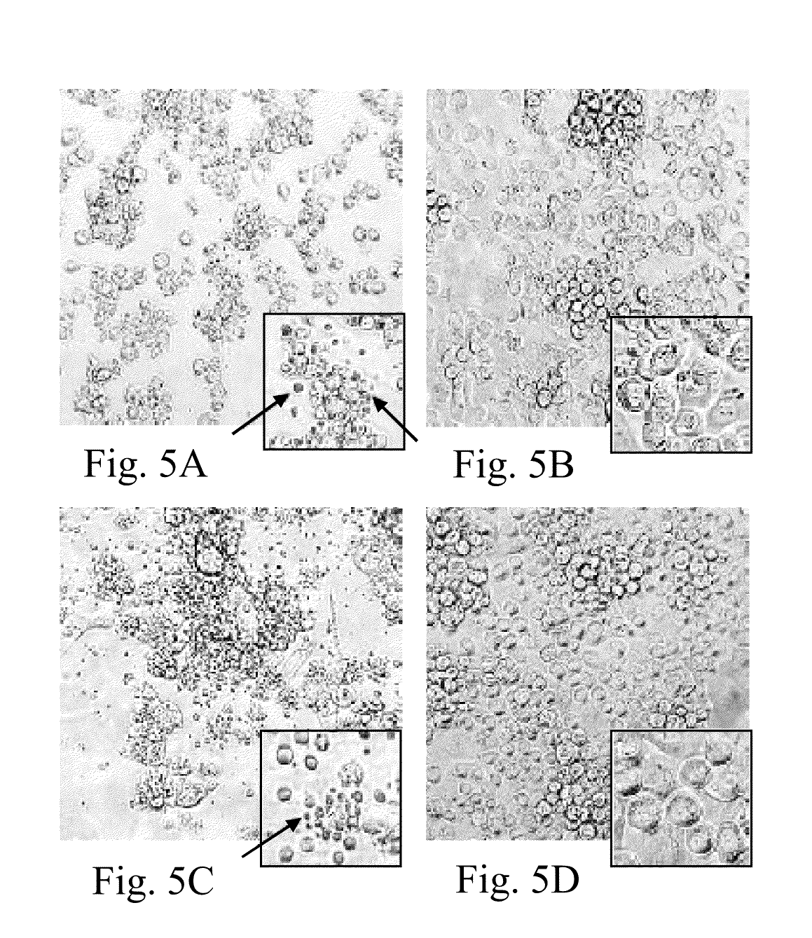

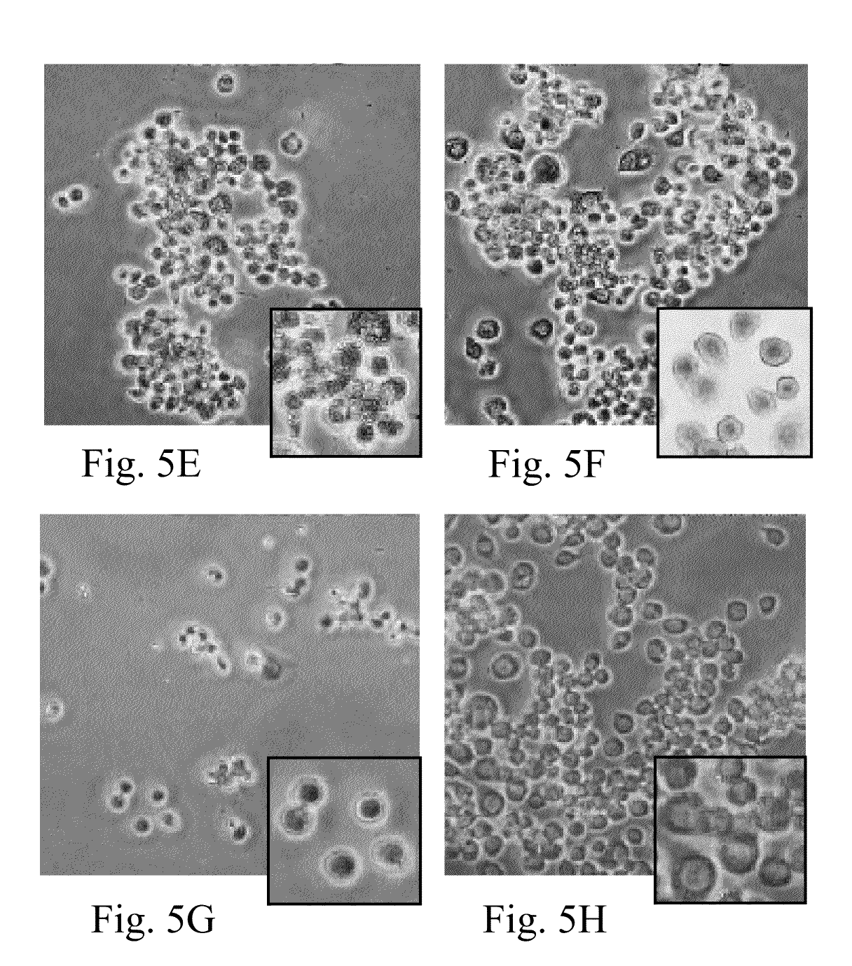

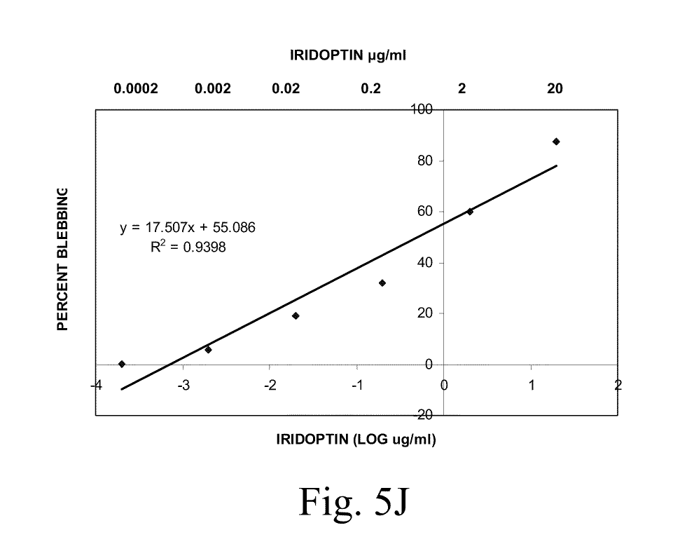

Reference is now made to FIGS. 5A-5D, wherein it is shown that iridoptin induces apoptosis in boll weevil cells (AG3A) as detected by cellular blebbing. AG3A cells were seeded in 60-well Terasaki plates and incubated at 28° C. for 24 hours. The highest dilution inducing blebs in 50% of the cell population was approximately 0.9 μg/ml. The blebbing assay shows in FIG. 5A that iridoptin at 20 μg/ml induced 87% blebbing in a boll weevil cell line, AG3A. FIG. 5B shows that heat-inactivated iridoptin (20 μg/ml, 65° C., 30 min) induced 7% blebbing. FIG. 5C shows that actinomycin D (4 μg/ml; positive control) induced 99% blebbing. FIG. 5D shows that mock treatment with RBSS buffer induced negligible blebbing (2.7%).

Reference is now made to FIGS. 5E-5H, wherein a TUNEL assay confirms iridoptin-induced apoptosis. FIG. 5E depicts AG3A cells treated with 10 μg/ml Iridoptin showed nuclear diaminobenzidine (DAB) signal confirming apoptosis in 53% of cell populations. FIG. 5F depicts AG3A cells treated with 20 μg/ml Iridoptin showed 84% nuclear DAB. FIG. 5G depicts AG3A cells treated with actinomycin D (ACT D) induced 96% nuclear DAB signal. FIG. 5H depicts AG3A mock treated with RBSS had 1% nuclear DAB.

Reference is now made to FIG. 5J, wherein a dose-response analysis of iridoptin-induced apoptosis against boll weevil cells (AG3A) as detected by blebbing is shown. The results show that 0.5 μg iridoptin is sufficient to induce apoptosis in 50% of the AG cell population. Similar results were obtained for CF cells. Results are demonstrated in FIG. 5J wherein the dose-response of iridoptin against boll weevil cells (AG3A) is shown according to percent blebbing. AG3A cells were seeded in 60-well Terasaki plates at 4.2×10³ cells per well, treated with serial 10-fold dilutions of iridoptin, and incubated at 28° C. for 24 hr. Actinomycin D (4 μg/ml) was the positive control and heat-inactivated iridoptin (20 μg/ml, 65° C., 30 min) was the negative control; mock treatments were with Rinadini's balanced salt solution (RBSS). Data points represent percent blebbing for approximately 200 cells per field. The highest dilution inducing blebs in 50% of the cell population was approximately 0.5 μg/ml. Linear regression line was generated using Microsoft Excel: y=17.5 log x+55, where x is the concentration of iridoptin in μg/ml and y is the percent of cell population with blebs; r²=0.94. Controls showed expected blebbing (actinomycin D: 99%, SD 1.5; heat-inactivated iridoptin: 7%, SD 1.7; RBSS: 3%, SD 1.9). Assays were performed in triplicate.

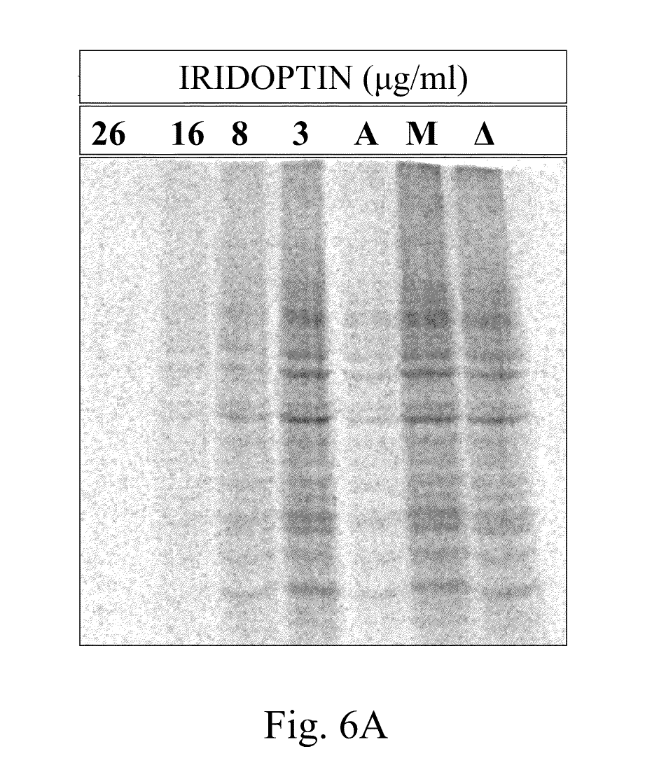

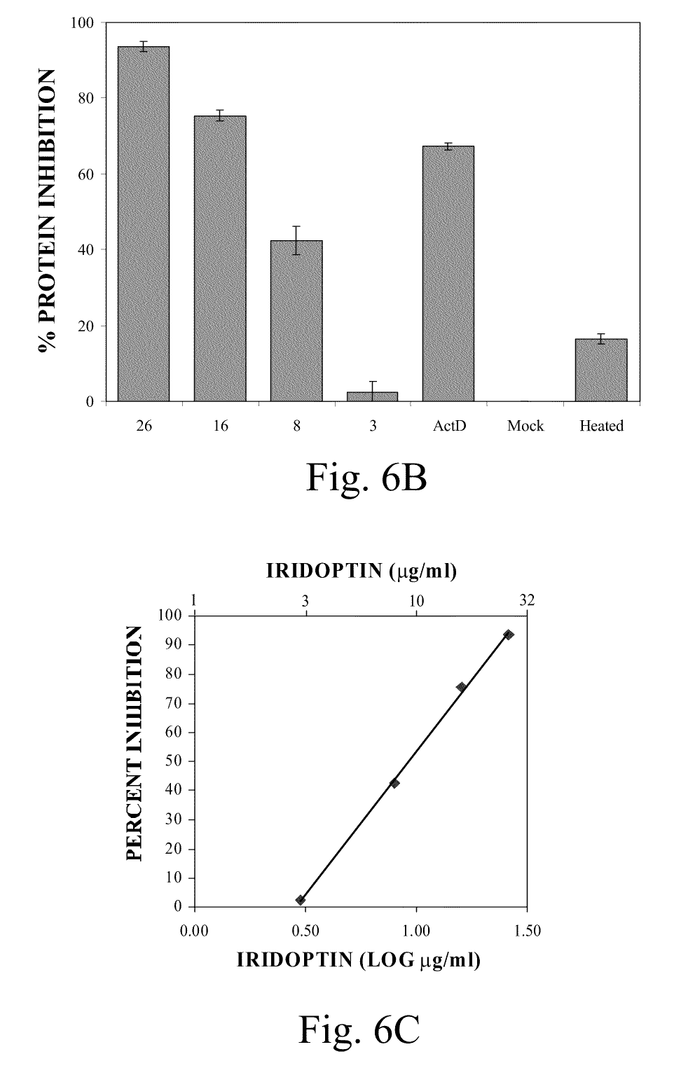

Reference is now made to FIG. 6A, wherein it is shown that iridoptin induced inhibition of protein synthesis in boll weevil (AG3A) cells. AG3A cells were seeded in Costar 24-well plates treated with iridoptin at the concentrations shown. Controls included actinomycin D (A, positive, 4 μg/ml), heat-inactivated iridoptin (A, negative, 10 μg/ml, 65° C., 30 min.), and mock (M, RBSS). Beginning at 3 hr post treatment, cells were pulsed with ³⁵S-methionine for one hour, lysed with SDS-PAGE sample buffer (2% SDS, 20% glycerol, 20 mM Tris-HCl pH 8, 2% β-mercaptoethanol, and 0.1 mg/ml bromophenol blue) and fractionated using SDS-PAGE. The relative rate of protein synthesis was quantitated using a phosphorimager. The experiment was performed in triplicate.

Reference is now made to FIG. 6B, wherein iridoptin-induced inhibition of protein synthesis in boll weevil (AG3A) cells is quantified. Iridoptin at 26 μg/ml inhibited more than 90% host protein synthesis, whereas actinomycin D inhibited 68% protein synthesis. AG3A cells were seeded in CoStar 24-well plates treated with iridoptin at concentrations (μg/ml.) shown in FIG. 6B. Controls included actinomycin D (ActD, positive control, 4 μg/ml), heat-inactivated iridoptin (Heated, negative control, 10 μg/ml, 65° C., 30 min), and cells that were mock-treated with Rinaldini's Balanced Salt Solution (Mock). Beginning at 3 hr post treatment, cells were pulsed with ³⁵S-methionine for one hour, lysed with SDS-PAGE sample buffer and fractionated using SDS-PAGE. Bands were visualized using a phosphorimager. The relative rate of protein synthesis was quantitated using functions of a phosphorimager. This experiment was performed in triplicate. All major bands from three independent experiments were used to determine inhibition. Iridoptin at 26 μg/ml inhibited more than 90% host protein synthesis.

Reference is now made to FIG. 6C, wherein a dose-response analysis of iridoptin-induced inhibition of protein synthesis in boll weevil (AG3A) cells is shown. The data from FIG. 6A was quantified using functions of the phosphorimager. The highest dilution to inhibit 90% host protein synthesis was approximately 23 μg/ml. Log of iridoptin concentrations was plotted against percent protein inhibition. A linear regression line was generated using Microsoft Excel 2003 with the following equation: y=98.4x−44.9, where x is log of iridoptin dilutions in μg/ml and y is percent inhibition of host protein synthesis. The r² value for the fitted line was 0.99.

Reference is now made to FIG. 7, wherein iridoptin-induced inhibition of protein synthesis in CF cells is shown. Inhibition with iridoptin (Iridoptin; 7 μg/ml; 63%) was 93% of that observed with the positive control, actinomycin D (Act D; 4 μg/ml; 68%); whereas inhibition with heated iridoptin (Δ Iridoptin; 7 μg/ml, 65° C., 30 min) was 18% of positive controls. The optical density of equivalent areas from relevant lanes in SDS-PAGE gels was measured using mock lanes as control and converted to percent transmittance. Inhibition values were determined from percent transmittance against mock lanes.

Reference is now made to Table 1, which depicts protein kinase activity of iridoptin. Assays for protein kinase showed significant activity for iridoptin. Gamma ³²P-ATP was used as label and protamine as substrate. Samples were spotted on phosphocellulose paper and radioactivity was counted after washing off excess label. Specific activity of kinase was expressed as cpm per μg total protein in the enzyme preparation used. The specific activity of iridoptin was slightly higher than that of CIV virion protein extract (CVPE). Kinase activity was low in heated iridoptin (65° C., 30 min), and BSA controls.

TABLE 1

| | SPECIFIC ACTIVITY (CPM/μg TOTAL PROTEIN) | | | |
|---|---|---|---|---|
| TREATMENT | BSA | IRIDOPTIN | Δ IRIDOPTIN | CVPE |
| REPLICATE 1 | 149 | 1113 | 250 | 889 |
| REPLICATE 2 | 74 | 895 | 223 | 958 |
| MEAN | 111.5 | 1004 | 236.5 | 923.5 |

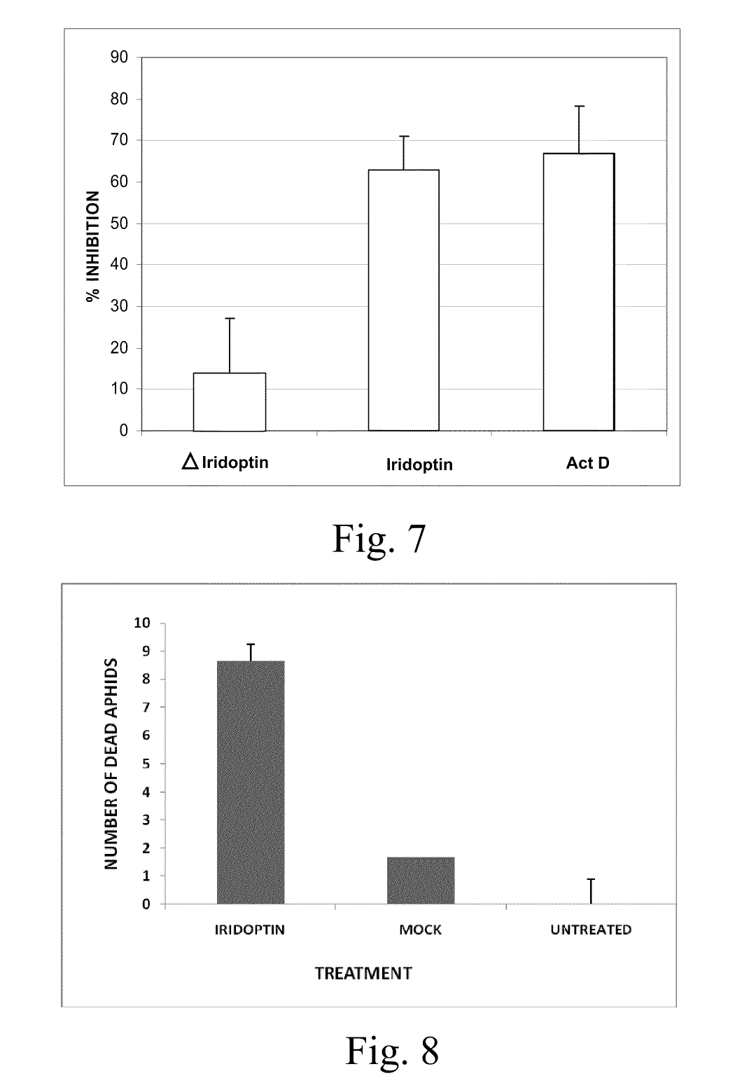

Reference is now made to FIG. 8, which depicts iridoptin induced mortality in aphids. Bioassays showed that iridoptin induced 72% mortality in treated aphids compared to 14% mortality in controls. The effect of iridoptin treatment on cotton aphids is shown in FIG. 8, wherein twelve aphids were placed on cotton leaves that were painted with the following preparations: 1) Iridoptin: the iridoptin gene was expressed in the Pichia system; yeast lysates were purified on nickel columns (ProBond), Eluates were desalted and exchanged with RBSS and diluted to 50 μg/ml with RBSS containing final concentrations of 20 μg/ml casein, 1 μg/ml pepstatin A, and 2 μg/ml leupeptin; 2) Mock: Pichia strain (X33) not containing the iridoptin gene was processed as above; lysates were mock-purified and diluted with the above solvents at ratios utilized for the iridoptin preparation; and 3) Untreated: aphids were incubated at 28° C. for 3 days and examined for mortality with a dissecting microscope. The results are presented as the number of dead aphids above that on untreated leaves. Aphid mortality in iridoptin-, mock-, and untreated leaves was 72%, 14% and 0%, respectively. The experiment was performed in triplicate. The bars indicate standard error.

The disclosed method and apparatus is generally described below, with the following examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to described specific embodiments of the invention, but their usage does not delimit the disclosed method, except as may be outlined in the claims.

TUNEL: A staining assay that detects fragmented DNA in the nuclei of apoptotic cells; positive stain is diagnostic for apoptotic cells.

Apoptosis: programmed cell death in which cells shrink, undergo nuclear DNA fragmentation, and develop blebs at the surface.

### EXAMPLES

By conducting tests of iridoptin for apoptosis activity, inhibition of host protein synthesis in cell culture, and mortality in aphids, it has been shown that iridoptin, the product of the modified istk gene from CIV, induces a very high level of apoptosis in more than 90% of treated insect cells, inhibits host protein synthesis, and kills 63% of aphid populations over control treatments. These data strongly suggest that iridoptin will have toxic or inhibitory effects against other insects, including the cotton boll weevil, lygus bug, the whitefly, and noctuids.

Alternate applications of this invention include using the DNA segment coding for iridoptin to engineer and produce: (i) cotton and other crop plants resistant to aphids, boll weevils, lygus bugs, the whitefly, noctuids and other insect pests, (ii) microorganisms for controlling agricultural pests as well as plant, animal, and human disease vectors and household pests, and (iii) large amounts of iridoptin for direct control of agricultural and household pests as well as disease vectors. By extension, Iridoptin finds application in cancer therapy and other medical treatments where apoptosis is critical to removal of certain cells.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention.

More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Bernal, J., González, D, E. T. Natwick, J. G. Loya, R. León-Lopez, W. E. Bendixen. (1993). “Natural enemies of Russian wheat aphid identified in California.” California Agric. 47: 24-28.
Bernal, J., González, D., Natwick, E. T., Loya, J. G., León-Lopez, R., Bendixen, W. E. (1993). California Agric. 47: 24-28.
Burkness, E. C., Hutchison, W. D., Weinzierl, R. A., Wedberg, J. L., Wold S. J., Shaw J. T. (2002). Crop Protection, 21(2): 157-169.
Cabrera, H. M., Argandoña, V. H., Zúñiga, G. E., Corcuera L. J. (1995). Phytochemistry 40(4): 1083-1088.
Carter, R. S., Stiebel, W.H., Nalasco, P. J., Pardieck, D. L. (1995). “Investigation and Remediation of Groundwater Contamination at a Pesticide Facility—a Case Study.” Water Quality Research Journal of Canada 30(3): 469-491.
Chitnis, N. S., D'Costa, S. M., Paul, E. R., and Bilimoria, S. L. (2008) Modulation of iridovirus-induced apoptosis by endocytosis, early expression, JNK, and apical caspase. Virology 370: 333-342.
D'Costa, S. M., H. J. Yao, et al. (2004). “Transcriptional mapping in Chilo iridescent virus infections.” Arch Virol 149(4): 723-42.
D'Costa, S. M., Yao, H., Bilimoria, S. L. (2001). Archives of Virology 146(11): 2165-78.
Funk, C. J., Hunter, W. B., Achor, D. S. (2001). J Invertebr Pathol. 77(2): 144-6.
Grossi-de-Sa M F, Quezado de Magalhaes M, M S Silva, S M Silva, S C Dias, E Y Nakasu, P S Brunetta, G R Oliveira, O B Neto, Sampaio de Oliveira R, L H Soares, M A Ayub, H A Siqueira, and E L Figueira (2007). Susceptibility of Anthonomus grandis (cotton boll weevil) and Spodoptera frugiperda (fall armyworm) to a crylia-type toxin from a Brazilian Bacillus thuringiensis strain. Journal of Biochemistry and Molecular Biology. 40:773-782.
Henderson, C. W., Johnson, C., Lodhi, S. A., and Bilimoria, S. L. (2001). “Replication of Chilo iridescent virus in the cotton boll weevil, Anthonomus grandis, and development of an infectivity assay.” Archives of Virology. 146: 767-775.
Henneberry, T. J. and R. M. Faust (2002). National Research, Action, and Technology Transfer Plan, 1997-2002: Fifth Annual Review of the Second 5-Year Plan.
Kazmierczak, J. R., F and B. C. Smith (1996). “Role of knowledge and opinion in promoting Boll Weevil (Coleoptera: Curculionidae) Eradication.” Journal of Economic Entomology 89: 1166.
Liang, G., Liu, T. X. (2002). “Repellency of a Kaolin Particle Film, Surround, and a Mineral Oil, Sunspray Oil, to Silver leaf Whitefly (Homoptera: Aleyrodidae) on Melon in the Laboratory.” J. Econ. Entomol. 95(2): 317-324.
Michels Jr., G. J. (2002). Precision Agriculture Initiative For Texas High Plains, 2002 Annual Comprehensive Report, 2002 Annual Comprehensive Report.
Norman Jr., J. W., Sparks Jr., A. N. (2003). Managing Cotton Insects in the Lower Rio Grande Valley, 2003 Texas Agricultural Extension Service Publication No. E-7.
Oerke, E. C., Dehne, H. W., Schonbeck, F., Weber, A. (1995). Crop Production and Crop Protection: Estimated Losses in Major Food and Cash Crops. Amsterdam, Elsevier Publishing Co.
Paul, E. R., Chitnis, N. S., Henderson, C. W., Kaul, R. J., D'Costa, S. M., and Bilimoria, S. L. (2007) Induction of apoptosis by iridovirus virion protein extract. Archives of Virology. 152: 1353-1364.
Pedigo, L. P. (2002). Entomology and Pest Management, Prentice Hall.
Westbrook, J., Suh, C., Jones, G., Spurgeon, D., Esequivel, J. (2002). Annual Report 2002 ARS Project Number: 6202-22000-007-00.
Westbrook, J., Suh, C., Jones, G., Spurgeon, D., Esequivel, J. (2002). Development of Area wide Management for Corn Earworm, Corn Rootworm & Other Field Crop Pests, Annual Report 2002 ARS Project Number: 6202-22000-007-00.
Williams, M. R. (2000). “Cotton Insect Losses 1999.” In Proceedings of the Beltwide Cotton Conferences: 887-913.
Williams, M. R. (2003). In (online) Proceedings 2003 Beltwide Cotton Production Conference.
Williams, T. (1998). Invertebrate Iridescent Viruses. The Insect Viruses. L. K. Miller and L. A. Ball. New York, Plenum Press: 31-68.

I claim:

1. An isolated nucleotide sequence encoding the complete istk gene as set forth in SEQ ID NO: 1 from nucleotide 46 through nucleotide 1275, wherein the istk gene product is a serine-threonine kinase enzyme 49-kDa polypeptide (ISTK).

2. The ISTK polypeptide of claim 1, wherein the polypeptide is capable of inducing apoptosis in insect cells or inhibiting protein synthesis in insect cells.

3. A subgenic fragment of the istk gene of claim 1, wherein the subgenic fragment has the nucleotide sequence as set forth in SEQ ID NO: 3.

4. The subgenic fragment of claim 3, wherein the subgenic fragment codes for a 37-kDa polypeptide (iridoptin) as set forth in SEQ ID NO: 2 from 1-290, and further wherein the polypeptide contains a serine-threonine kinase enzyme.

5. The iridoptin polypeptide of claim 4, wherein the polypeptide exhibits enhanced toxicity to insects as compared to ISTK.

6. A method of producing a 37-kDa polypeptide (iridoptin) as set forth in SEQ ID NO: 2 from 1-290, the method comprising the steps of:
(a) providing an isolated nucleotide sequence encoding the complete istk gene as set forth in SEQ ID NO: 1 from nucleotide 46 through nucleotide 1275, wherein the istk gene product is a serine-threonine kinase enzyme 49-kDa polypeptide (ISTK);
(b) identifying the cleavage site on the ISTK polypeptide;
(c) identifying the C-terminal amino acids;
(d) detecting the C-terminal amino acids in the amino acid sequence derived from the istk gene; and
(e) cleaving the ISTK polypeptide to yield the 37-kDa polypeptide (iridoptin) containing both ATP binding site and serine-threonine kinase enzyme.

7. A method of isolating a subgenic fragment of the istk gene as set forth in SEQ ID NO: 3, wherein the subgenic fragment encodes the polypeptide of claim 4, the method comprising:
(a) performing C-terminal sequencing of the 37-kDa polypeptide (iridoptin) as set forth in SEQ ID NO: 2 from 1-290;
(b) confirming the molecular weight and N-terminal sequencing of the 37-kDa polypeptide;
(c) effecting a nucleic acid amplification reaction of the subgenic DNA sequence coding for the 37-kDa polypeptide using specifically designed primers and Chilo iridescent virus genomic DNA; and
(d) expressing the product utilizing the Pichia system.

8. A vector comprising a polynucleotide sequence encoding the polypeptide as set forth in SEQ ID NO: 2 from 1-290, wherein the polypeptide exhibits enhanced toxicity to insects as compared to Chilo iridescent virus capsid protein extract.

9. A host cell comprising a polynucleotide sequence encoding the polypeptide as set forth in SEQ ID NO: 2 from 1-290, wherein the polypeptide exhibits enhanced toxicity to insects as compared to Chilo iridescent virus capsid protein extract.

10. The host cell according to claim 9, wherein the cell is a plant cell or a bacterial cell.

11. The cell according to claim 10, wherein the plant cell is a cotton cell.

12. A method of producing a transformed cell, the method comprising: introducing into a cell a subgenic fragment of the istk gene encoding a 37-kDa polypeptide (iridoptin) that has the SEQ ID NO: 2 from 1-290, and wherein the cell is a plant cell or a microbial cell.

13. The method of claim 12, wherein the cell is a plant cell.

14. The method of claim 13, wherein the plant cell is a cotton cell.

15. A method of producing a transformed cell, the method comprising: introducing into a cell the nucleotide sequence as set forth in SEQ ID NO: 3, and wherein the cell is a plant cell or a microbial cell.

16. A method of producing an insect resistant plant, the method comprising the steps of:
(a) introducing into a plant cell the nucleotide sequence as set forth in SEQ ID NO: 3, for expression of an insecticidal protein;
(b) selecting a transformed plant cell; and
(c) regenerating a plant from the transformed plant cell, wherein the plant comprises the nucleotide sequence.

17. A seed or progeny from a plant produced according to the method of claim 16, wherein the seed or progeny comprises the nucleotide sequence.

18. A plant comprising the nucleotide sequence as set forth in SEQ ID NO: 3.

19. A seed or progeny from the plant of claim 18, wherein the seed or progeny comprises the nucleotide sequence.

20. A plant grown from the seed of claim 19.

21. A method of controlling an insect infestation in a field of crop plants, the method comprising: providing to an insect a transgenic plant on which the insect feeds, the transgenic plant expressing the insecticidal protein encoded by the polynucleotide sequence as set forth in SEQ ID NO: 2 from 1-290.

22. A method of controlling an insect infestation of a plant, the method comprising: providing to an insect a transgenic plant on which the insect feeds, the transgenic plant expressing the insecticidal protein encoded by the polynucleotide sequence as set forth in SEQ ID NO: 2 from 1-290.

23. An insect-controlling composition, comprising a suitable carrier and an insect-controlling amount of an isolated insecticidal Chilo iridescent virus protein comprising a polynucleotide sequence encoding the polypeptide as set forth in SEQ ID NO: 2 from 1-290, wherein the polypeptide exhibits enhanced toxicity to insects as compared to Chilo iridescent virus capsid protein extract.

24. A method of controlling insect pests or insect disease vectors that infest or transmit disease among plants, humans, or animals, comprising: applying to the location wherein the insect is to be controlled an insect-controlling amount of an isolated insecticidal Chilo iridescent virus protein comprising a polynucleotide sequence encoding the polypeptide as set forth in SEQ ID NO: 2 from 1-290, wherein the polypeptide exhibits enhanced toxicity to insects as compared to Chilo iridescent virus capsid protein extract.

25. The method of claim 24 wherein the polypeptide is ingested by the insect or introduced to the insect by contact.

26. The method of claim 24 wherein the insect is sprayed with the polypeptide.

27. The method of claim 24 wherein the insect is an aphid or other member of the order Homoptera.

28. The method of claim 24 wherein the insect is selected from the group consisting of boll weevil, lygus bug, white fly, and noctuid.

29. The method of claim 24 wherein the location is a plant.

30. The method of claim 29 wherein the plant is selected from the group consisting of cotton, maize, alfalfa, rape, bean, potato and rice plants.
